(19) United States
(12) Patent Application Publication
(10) Pub. No.: US 2026/0002199 A1
(43) Pub. Date: Jan. 1, 2026

(54) MULTILAYERED PLANAR SENSOR FOR DETECTION OF MATERIALS INCLUDING SPECIFIC DNA OR DNA-LIKE STRANDS

(71) Applicant: DNA PROFILE LTD., Tel-Aviv (IL)

(72) Inventor: Roberto Igal CHERTKOW, Ashdod (IL)

(21) Appl. No.: 19/132,782

(22) PCT Filed: Nov. 7, 2023
(86) PCT No.: PCT/IL2023/051147

§ 371 Date: May 23, 2025

Related U.S. Application Data
(60) Provisional application No. 63/428,087, filed on Nov. 27, 2022.
(60) Provisional application No. 63/435,258, filed on Dec. 25, 2022.

(51) Int. Cl. C12Q 1/6825

(52) U.S. Cl. CPC C12Q 1/6825

(57) ABSTRACT

A multilayered sensor device for detecting DNA or DNA-like strands within a biological sample includes: a planar electret layer having a positive pole; a sensing layer configured above the electret layer including a plurality of sensors comprising pairs of conductive elements with gaps therebetween, wherein strands of single-stranded DNA of the sensor are configured within the gaps, wherein the strands of single-stranded DNA of the sensor within the gaps are configured to couple with strands of single-stranded DNAfrom the biological sample; and a solution layer configured above the sensing layer, said solution layer comprising chemical solutions for breaking down a biological sample into DNA strands. The positive pole is arranged relative to the sensing layer and solution layer such that, following breakdown of the biological sample into DNA strands, the positive pole draws strands of single-stranded DNA through the solution layer and to the sensing layer.

### RELATED APPLICATIONS

This Application claims priority to U.S. Provisional Patent Application No. 63/423,087, filed Nov. 27, 2022, entitled “Passive Sensor Capable of Detection of Materials Including Specific DNA or DNA-Like Strands,” and to U.S. Provisional Patent Application No. 63/435,258, filed Dec. 25, 2022, entitled “Passive Sensor Capable of Detection of Materials Including Specific DNA or DNA-Like Strands,” the contents of which are incorporated by reference as if fully set forth herein.

### FIELD OF THE INVENTION

The present invention relates to the sensing of biological and DNA-like molecules. More particularly, the invention relates to a sensor capable of recognizing different strands or strand fragments of cDNA/DNA, or RNA, which operates passively without requiring an internal source of power. In particular, the present invention relates to a multilayered planar implementation of such a sensor.

### BACKGROUND OF THE INVENTION

Various methods are available for the detection of DNA-like molecules. Such methods include, gel electrophoresis and binding the DNA to DNA-specific fluoresecent dyes. These methods generally require transporting the sample to a laboratory with dedicated equipment and highly qualified personnel. These methods are too inefficient and too expensive to be useful on a mass scale.

As personalized treatments are developed for people with specific DNA sequences, it is becoming increasingly desirable to detect specific DNA sequences within particular people. For example, in the fields of microbiome and personalized beauty, capabilities are being developed to enable matching between the genomic properties of an individual and food or cosmetic products that are best adapted to him or her.

There is accordingly a need for a simple, low-cost, disposable, passive sensor, which does not require an internal power source, for detecting cDNA/DNA/RNA like molecules.

In addition, there is a need for an implementation of this sensor that is low-cost, easy to manufacture, and relies on passive principles for isolating the cDNA/DNA/RNA-like molecules from the biological sample from which they were obtained.

### SUMMARY OF THE INVENTION

The present disclosure teaches a sensor capable of conducting tests of a material in general and RNA/DNA/DNA-like molecules in particular, without the involvement of highly trained, expensive personnel using costly laboratory equipment. The sensor is simple, cheap, and disposable. In addition, the sensor does not require an on-sensor power supply, complex electronic microchips, a microprocessor, multiplexers and complex receivers-transmitters on the testing device. The device may be implemented, inter alia, in the fields of microbiome and personalized beauty, to enable matching between the genomic properties of an individual and food or cosmetic products that are best adapted to that individual.

In particular, the present disclosure teaches a multilayered, substantially planar sensor device. The multilayered sensor device includes a central sensing layer with a sensor element for detecting the presence of the DNA or DNA-like molecules. The sensor device further includes one or more upper layers for breaking up a biological sample and extracting DNA strands therefrom. In addition, the sensor device includes an electret layer below the sensing layer, for drawing the extracted DNA strands to the sensing layer, and a reservoir layer below the sensing layer, for collecting materials that pass through the sensing layer. The entire sensor may be arranged on a planar substrate which may be rigid or flexible.

The present disclosure further teaches a method of manufacture of the multilayered, planar sensor device. In particular, the sensor device may be manufactured using a roll-to-roll technology, in which the layers are laminated one on top of the other. During this manufacturing process, multiple sensor devices may be manufactured on a roll of substrate material, and the sensor devices may then be separated following completion of the layering process. The sensor devices may thus be prepared efficiently and at low cost.

According to a first aspect, a multilayered sensor device for detecting DNA or DNA-like strands within a biological sample is disclosed. The device includes: a substantially planar electret layer, the electret layer having a positive pole that is configured to attract negatively charged particles; a sensing layer configured above the electret layer, the sensing layer including a plurality of sensors comprising pairs of conductive elements with gaps therebetween, wherein strands of single-stranded DNA of the sensor are configured within the gaps, wherein the strands of single-stranded DNA of the sensor within the gaps are configured to couple with strands of single-stranded DNA from the biological sample; and a solution layer configured above the sensing layer, said solution layer comprising chemical solutions for breaking down a biological sample into DNA strands. The positive pole is arranged relative to the sensing layer and solution layer such that, following breakdown of the biological sample into DNA strands in the solution layer, the positive pole of the electret layer draws strands of single-stranded DNA of the sample through the solution layer and to the sensing layer.

In another implementation according to the first aspect, the device includes a separation layer between the sensing layer and the electret layer. Optionally, the separation layer is permeable to liquids and comprises a dump reservoir for liquids that are attracted to the electret layer but are not coupled to the single-stranded DNA of the sensor.

In another implementation according to the first aspect, each pair of conductive elements comprises plates of a capacitor, with the single-stranded DNA of the sensor configured between the plates of the capacitor. Optionally, the single-stranded DNA of the sensor is arranged on beads, and preferably conductive beads configured between the plates of the capacitor. Optionally, the conductive beads are arranged with voids therebetween, to permit passage therethrough of biological material from the sample that does not couple to the single-stranded DNA. Optionally, the voids are filled with one or more of air, gases, liquid, or gels.

In another implementation according to the first aspect, each pair of conductive elements comprises optical waveguides. The single-stranded DNA of the sensor is arranged in gaps between the waveguides.

In another implementation according to the first aspect, the sensor device further includes a substrate layer arranged below the electret layer, wherein the substrate layer is non-conductive and liquid-resistant. Optionally, the sensor device further includes a sensor sealing layer configured between the sensing layer and the solution layer, the sensor sealing layer comprised of material that is impervious to liquids and including a central opening therein opposite the single-stranded DNA of the sensing layer. Optionally, the opening is filled with one or more of a non-conducting liquid, a gel, or a compressed powder.

In another implementation according to the first aspect, the solution layer comprises a plurality of sheets stacked one over the other, with each sheet containing a plurality of solutions configured to perform specific chemical functions on the biological material. Optionally, the plurality of sheets includes an uppermost layer containing a protein kinase solution; a second layer below the uppermost layer containing a restriction enzyme solution, and a third layer below the second layer containing a dissociating solution.

According to a second aspect, a method of drawing single-stranded DNA strands to a sensor for sensing the presence of specific single-stranded DNA strands is disclosed. The sensor comprises a substantially planar electret layer, the electret layer having a positive pole that is configured to attract negatively charged particles; a sensing layer configured above the electret layer, the sensing layer including a plurality of sensors comprising pairs of conductive elements with gaps therebetween, wherein strands of single-stranded DNA of the sensor are configured within the gaps, wherein the strands of single-stranded DNA of the sensor within the gaps are configured to couple with strands of single-stranded DNA from the biological sample; and a solution layer configured above the sensing layer, said solution layer comprising chemical solutions for breaking down a biological sample into DNA strands. The method comprises: breaking down the biological sample into single-stranded DNA of the sample in the solution layer; and drawing the single-stranded DNA of the sample from the solution layer to the sensing layer with the positive pole of the electret layer.

In another implementation according to the second aspect, the method further includes coupling strands of single-stranded DNA from the sample with strands of single-stranded DNA of at least one sensor, and measuring resulting changes of electrical properties of the at least one sensor.

According to a third aspect, a method of manufacturing a multilayered sensor device is disclosed. The method includes: providing a substrate layer; cutting a plurality of sheets of electret material, and laminating the plurality of sheets of electret material onto the substrate layer to thereby form a plurality of electret layers; heating and applying an electric field to each electret layer to thereby pole the electret material; laminating a sensing layer onto each separation layer, the sensing layer including a plurality of pairs of conductive elements with gaps therebetween; configuring strands of single-stranded DNA of the sensing layer within the gaps; and laminating a solution layer above the sensing layer, the solution layer comprising chemical solutions for breaking down a biological sample into DNA strands of the sample.

In another implementation according to the third aspect, the step of providing a substrate layer comprises providing the substrate layer in the form of a roll and unrolling the roll, and wherein the laminating steps are performed on the unrolled roll.

In another implementation according to the third aspect, the method further includes performing each of the laminating steps using a roll-to-roll process.

In another implementation according to the third aspect, each laminating step comprises laminating respective layers of multiple multilayered sensor devices simultaneously, and further comprising, following completion of the laminating steps, separating the multilayered sensor devices.

### BRIEF DESCRIPTION OF THE DRAWINGS

FIGS. 1A-1C illustrate layers of a multilayered sensor, according to embodiments of the present disclosure;

FIGS. 2A-2B illustrate the function of an electret layer within the multilayered sensor, according to embodiments of the present disclosure;

FIGS. 3A-3B illustrate movement of DNA strands through the layers of the multilayered sensor of FIGS. 1A-1C, according to embodiments of the present disclosure; and

FIGS. 4A-B illustrate steps in roll-to-roll manufacturing of the multilayered sensor, according to embodiments of the present disclosure.

### DETAILED DESCRIPTION OF THE INVENTION

The present invention relates to the sensing of biological and DNA-like molecules. More particularly, the invention relates to a sensor capable of recognizing different strands or strand fragments of cDNA/DNA, or RNA, which operates passively without requiring an internal source of power. In particular, the present invention relates to a multilayered planar implementation of a sensor device containing such a sensor.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways. In particular, throughout this disclosure, when the disclosure described that an element “may” be present, it is understood that the described element is not necessarily present, and the element may be replaced with an equivalent element or not be present at all. Likewise, when a list of specific examples is given, the list is not necessarily exclusive, even without a disclaimer such as “including but not limited to,” and other suitable examples may be utilized.

The sensor device described herein, in certain embodiments, is configured for sensing of DNA or DNA-like samples. Throughout the remainder of this disclosure, the analyte will be described as “DNA,” with the understanding that the sensor device described herein may be used for sensing other materials generally, and specifically other types of DNA-like materials, such as RNA. The principles described herein are also applicable for isolating any biological or non-biological material for sensing, with appropriate modifications. In advantageous embodiments, the DNA samples are collected from a bodily sample, for example, saliva or a cheek swab. The sensor device may be used for detection of specific DNA or DNA-like strands (e.g., RNA), whether of cells of a person, of the microbiome, or of a virus. The DNA samples are then isolated, through the action of chemical solutions, within the sensor device, and brought to a sensing element within the sensor device.

In particular embodiments, the sensors described herein are configured for sensing specific single-stranded DNA strands). The principle of operation of these sensors is described at length in the aforementioned priority applications, which are incorporated by reference as if fully set forth herein. In brief, the sensors each contain one or more specific synthetic strands of single-stranded complementary DNA (sscDNA). When strands of ssDNA from a sample come into contact with the sscDNA of the sensor, they hybridize to the single-stranded DNA of the sensor to create double stranded DNA (dsDNA). The sensor is configured to measure differences in physical properties that result from the hybridization of the sscDNA. For example, the ssDNA of the sensor may be configured between plates of a capacitor that is part of a resonance circuit. Bonding of the ssDNA of the analyte to the sscDNA of the sensor causes a change in capacitance of the capacitor, and, as a result, a change in the resonance frequency of the resonance circuit. The resonance circuit may act as a band stop filter for particular frequencies sent and received by a transmitter-receiver. In alternative embodiments, the single-stranded DNA of the sensor is configured between adjacent waveguides of an optical coupler, and the bonding of the ssDNA of the analyte to the sscDNA of the sensor changes the wavelengths of light that are able to be coupled across the optical coupler. The sensor device and the system on which it dwells further includes means (e.g., detectors) that are able to monitor these physical changes.

In order to ensure that the sensor is able to effectively distinguish between solutions in which the analyte DNA is not present, versus solutions in which the analyte DNA is present but is unable to reach the sensor DNA, it is preferable to isolate the analyte DNA from other materials. This may be accomplished by various techniques, such as gel electrophoresis. However, many such techniques require expensive laboratory equipment and trained technicians. The present disclosure introduces a multilayer, substantially planar device that is capable of isolating ssDNA from a biological sample. This device includes various solutions for breaking down a biological sample (e.g., saliva or cheek swab) into ssDNA, and for isolating the DNA strands from the rest of the biological material.

The multilayer device further includes means for drawing the analyte DNA to the sensing element of the device, so that the analyte DNA is preferentially transferred from each layer to the next layer, while the other material from the biological sample remains behind. In examples illustrated herein, these means are electrets. As used in the present disclosure, an electret is a material into which a pole is introduced, such that into one of its surfaces a positive charge arises, and, if designed so, at the opposite surface a negative charge originates. In general, an electret is prepared from a sheet of polymer by applying a high voltage to the polymer film while the film is being heated, so that the molecules shift, thus creating the charge distribution, however other techniques are known in the art to create or pole electrets as well. Because a DNA molecule has inherent negative charge, a positive pole of the electret attracts the DNA molecule, while a negative pole of the DNA molecule repels the DNA molecule. Alternatively, a single-poled material with positive charges may be used to attract the negatively charged species and bring them into contact with the attached molecules in the sensing zone.

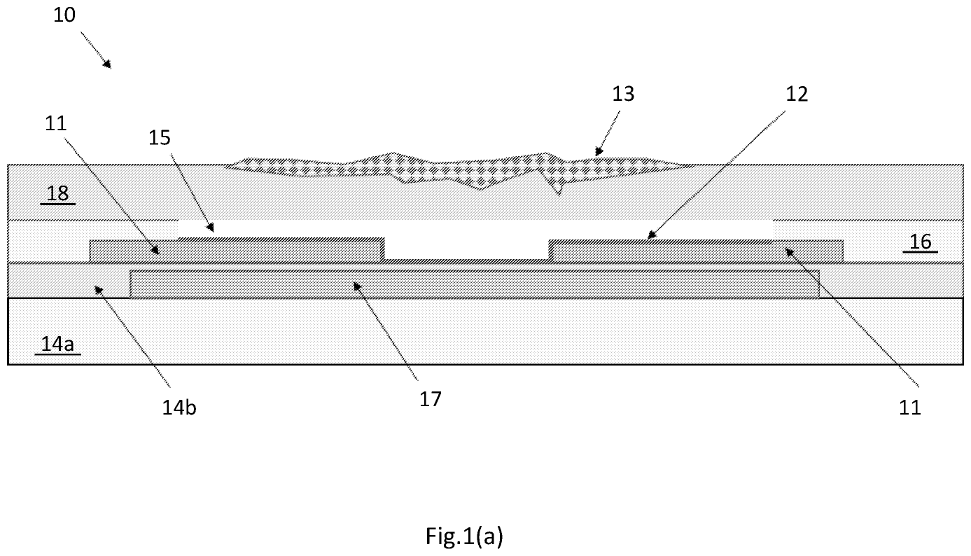

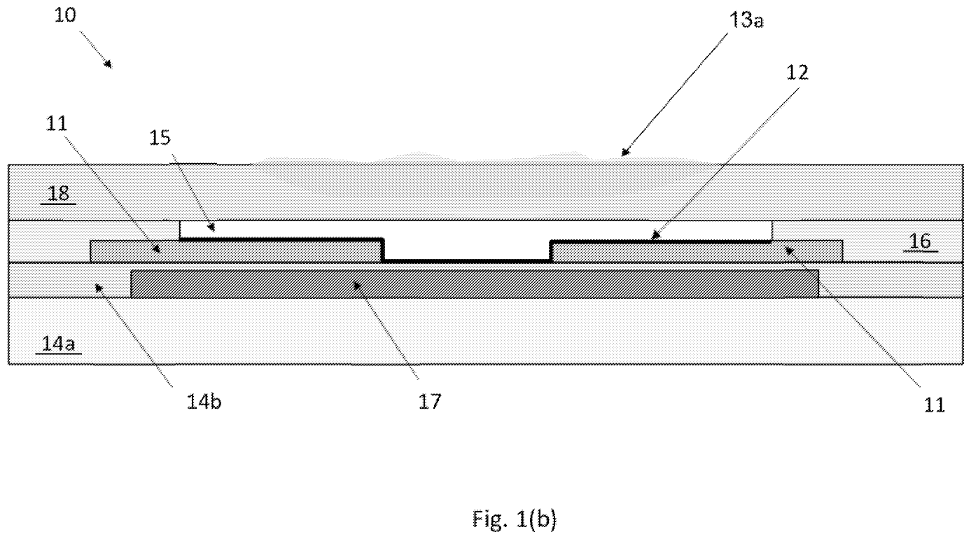

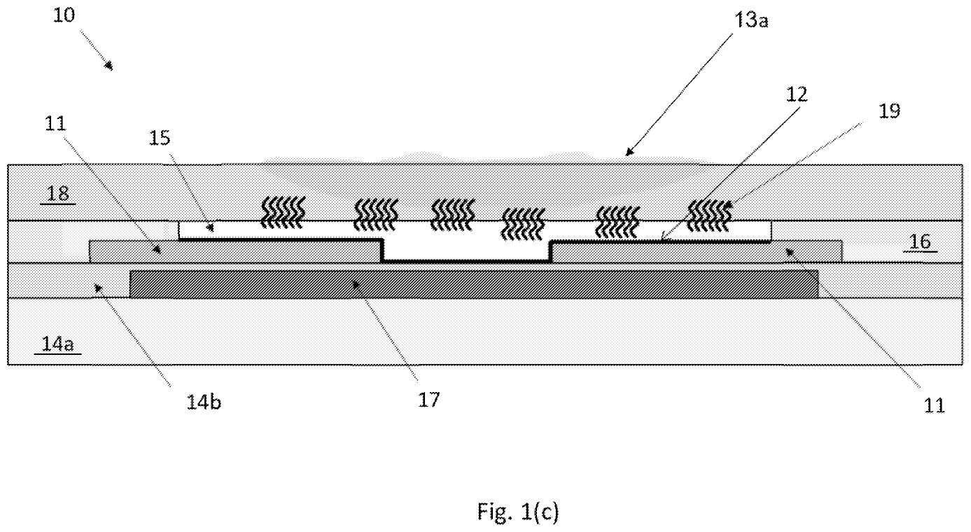

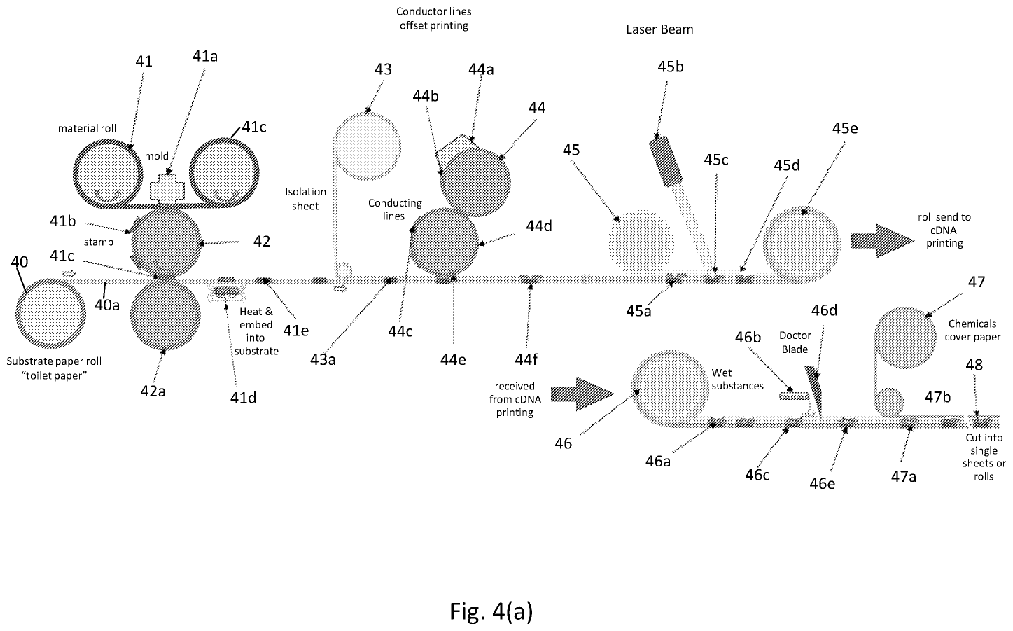

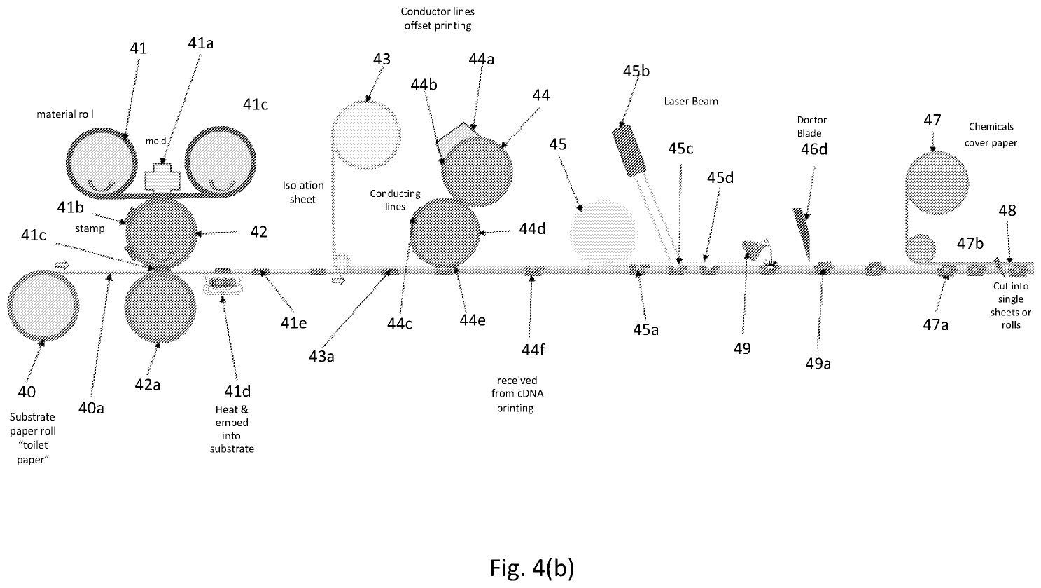

FIGS. 1A-C, 2A-B, and 3A-B illustrate the various layers of a multilayered planar sensor for extracting DNA from a biological sample, bringing the extracted DNA to a sensing element, and using the sensing element to determine the presence of specific DNA or DNA-like strands. FIGS. 4A-B disclose methods of manufacture of the multilayered planar sensor.

FIG. 1A schematically illustrates elements of a multilayered planar sensor 10. The multilayered planar sensor 10 is also referred to herein as a “smart wipe,” because it may resemble a cleaning wipe in structure and texture. Sensor 10 includes, in a sheet form, various chemical, electrical, and transport elements required for isolation and detection of specific DNA strands. Sensor 10 may be used for detection of specific DNA or DNA like-strands, whether of cells of a person, of the microbiome, or of a virus.

FIG. 1A illustrates schematically a cross-section view of multilayered planar sensor device 10. Sensor device 10 is composed of several layers that are laminated, glued, connected, or otherwise placed in close connection between each other. In the assembled state, the sensor device resembles a paper-like structure of several layers.

Sensor device 10 optionally includes a bottom layer that is a substrate 14a. Substrate 14a, among other functions, may hold the entire structure of sensor device 10 on top of it. Substrate 14a may be made of paper, a polymer, or of any other suitable material. Preferably, substrate 14a is non-conductive and liquid-resistant.

On top of substrate 14a, a sheet of electret material 17 is placed. Electret 17 may be attached to substrate 14a through any suitable method, such as bonding or laminating. The electret material 17 is poled, so that it is able to attract negatively-charged species. The process of poling the electret is described further herein. The electret may occupy the entire surface over substrate 14a. Alternatively, as shown in FIG. 1A, the electret 17 may be patterned, so that it is placed only over strategic zones of the sensors.

On top of electret 17, a second isolation or spacer sheet 14b may be placed and attached, such as by being bonded or laminated. Sheet 14b may be used for various purposes. These include: a separation between the electrical components of the sensor device and the electret 17; a holding reservoir for unused material; or for structural needs. Layer 14b may be made of the same material as substrate 14a or of different material. It may be permeable to liquids or may be impermeable to liquids, as needed.

On top of these layers, a sensing layer is implemented. The sensing layer includes various elements which might be in the electrical, optical, or other technical domains. For example, the sensing layer may contain a coupling inductor, a waveguide configured to propagate an electromagnetic wave signal from the coupling inductor, and one or more RF filters, having inductive elements and capacitive elements. The specific functioning of the sensing element is described at length in the priority applications referenced above.

The components of the sensing element may be made of conductive materials, such as metals such as gold or aluminum, doped polysilicon, conductive polymers, or conductive pastes and epoxies. These conducting elements, and, in general, the sensing layer may be prepared of one or more conducting layers separated by non-conducting material, the layers being connected by conducting vias. A via is a conducting connection between a conducting layer and a different conducting layer separated by a nonconducting layer, as it is well known in the art. In preferred embodiments, the sensing layer is formed of a single layer of conducting material.

In the specific embodiment of FIG. 1A, the conductive layer is shown as two electrodes 11 of a capacitor of a resonator of a sensing device. Dielectric materials are configured at least between the electrodes 11. The conductive elements are embedded from above with sheet 16 of spacing material. Layer 16 is preferably made non-conductive, so as to prevent short-circuiting of the electric circuit below. Sensor sealing layer 16 is patterned so as to have an opening over the electrodes 11 at the sensing zone where least part of the dielectric materials reside. Since the analyte is preferably delivered to the sensing device through a liquid, the material of this layer 16 is preferably impermeable to the liquid substances. The dielectric material in some cases includes single-stranded DNA (ss-DNA) or ss-DNA-like fragments 12 which are attached in the opening, at the exposed surfaces and particularly on the surfaces of the electrodes 11 and the gap between the electrodes 11 mostly on the substrate surface 14b or other surfaces present in the gap such as beads etc. These fragments are also referred to herein as “single-stranded DNA of the sensor.” The opening may be filled with a material 15 which permits transfer of the material to be tested toward the attached ssDNA-like molecules 12. Material 15 ideally has a minimal impact on the capacitance between electrodes 11, and may be, for example, a non-conducting liquid such as an oil or deionized water, a gel, or compressed powder.

In alternative embodiments, instead of being comprised of capacitive elements, the sensor may be comprised of optical waveguides. In such embodiments, elements 11 may be construed as adjacent optical waveguides, with the central opening between elements 11 being the gap across which light in one waveguide couples to the second waveguide and accordingly may transfer from the first to the second waveguide.

Attachment or coupling of single-stranded DNA of the sample to single-stranded DNA of the sensor causes a measurable physical change in the sensor element. For example, when the sensor element includes single-stranded DNA between capacitive elements, the capacitive elements may be included within a resonant circuit. Coupling of single-stranded DNA of the sample to single-stranded DNA of the sensor influences the frequency of the resonance. The resulting frequency of resonance is measurable, as is described at length in the aforementioned priority applications. As another example, when the sensor element includes single-stranded DNA in a gap between optical waveguides, coupling of single-stranded DNA of the sample to single-stranded DNA of the sensor influences the frequencies of light that traverse the gap. The resulting frequencies that are able to traverse the gap are measurable, as is described at length in the aforementioned priority applications.

For ease of reference, for the remainder of this disclosure, the sensing elements are described as part of a capacitor, with the understanding that the same disclosure applies equally to sensing elements that are coupling optical waveguides.

Layer 18 is configured above the sensing layer. Layer 18 is also referred to herein as a solution layer. This layer is made of a permeable material that can hold liquids, and is porous enough to allow the transfer of the biological samples as required. This layer is intended to contain and be soaked with all the chemical substances and solutions required for the isolation of the single-stranded DNA from the sample, as will be explained infra.

It is understood by those of skill in the art that the layers described herein are provided for best understanding of the idea, but in actual devices not all the layers need be present, and furthermore additional layers may be present as well. It is further understood that although the multilayered planar sensor is described as a “smart wipe,” and it is insinuated that the structure is thin and flexible like a sheet of paper, it need not be so, and the sensor device may also be made rigid or semi-rigid, and can take any form or structure as might be desirable.

Having described the structure of the device 10, the functioning of the device 10 will now be explained. Referring to FIG. 1A, a sample 13, such as a secretion or saliva or cheek swab taken from an individual, is placed on the smart wipe at the solution layer 18. Sample 13 penetrates into solution layer 18.

Referring to FIG. 1B, when the sample 13 makes contact with layer 18, the solutions present therein start to dissolve and break the boundaries of the cells and/or other biological components that might be in the sample. For example, the solutions present in layer 18 may include protein kinase, for breaking down cell walls. As a result, DNA molecules 13a present in the cells or components of the sample are released. Once the DNA molecules 13a are extracted, additional solutions present in the upper wet layer (e.g., restriction enzymes) separate the DNA and cut the DNA into fragments. The fragments are further separated into single strands, through action of solutions present in the solution layer 18 (e.g., NaOH or other dissociating solutions).

Referring to FIG. 1C, many ssDNA fragments 19 will be present at this stage on the layer 18. The DNA fragments have inherent negative charges and are drawn to the positively charged electret layer 17. Therefore, upon being released, the molecules will drift through layers of the sensing device 10, eventually encountering different solutions that may cut the double-stranded DNA into smaller fragments of DNA, and split the double-stranded DNA into single-stranded DNA at predefined locations and dimensions as needed. These fragments are also referred to herein as “single-stranded DNA of the sample.” This charged material drifts, through action of electret 17, toward the sensing region, where the attached ssDNA fragments 12 may bond to the incoming material. In case the incoming material, i.e., fragments 19, matches the existing material and bonds, this will prevent these fragments from proceeding to drift. All other charged material will continue to drift and eventually accumulate, whether in layer 18 for non-charged or positively charged components, or in separation layer 14b for those negatively charged components. As the attached fragments 12 and the incoming fragments 12 bond, they change the dielectric constant of the combined material between electrodes 11, and thus the capacitance of the sensor. This effect on the capacitance is detectable through measurement of a resonance frequency of a circuit which includes the capacitor. If, however, no matching fragment exists, no fragment will bond, and no identifiable change will occur.

In the illustrated embodiment, the solution layer 18 is illustrated schematically as a single sheet or layer which may contain all of the solutions needed for the chemical reactions to occur. This might not always be the case, and the layer 18 might be subdivided into several sheets, stacked, one over the other, with each layer containing different or similar solutions as needed to perform tasks on the biological material 13 being applied at the upper external layer of the stack. For example, the plurality of sheets may include an uppermost layer with protein kinase for breaking down cell walls, a second layer with restriction enzymes for dividing DNA strands, and third layer including dissociating solutions for splitting dsDNA into ssDNA. Thus, the reactions and splits previously described may occur at each layer separately and the species thus released or created will move lower within the sheet for further processing. This movement proceeds, at least in part, due to the action of the electret 17 on the charged molecules being released and/or created in the solutions. It is also clear that larger molecules will move more slowly than smaller molecules, so that a preference of motion will occur with respect to the smaller molecules, after being reacted and split, in their motion toward the sensing area. In addition, between the layers previously mentioned, either at the solution layer 18 and its components or between it and the sensing layer or at the sensing layer or between the sensing layer and the separation layer or any other layers, it is possible to add a filtering layer (not shown) that allows transfer therethrough of particles or molecules of certain dimensions or qualifications, such that only the required molecules or particles will transit to the next level as might be needed. As a result, less unintended material will arrive to the sensing area, which further eases the detection of the intended sample.

In addition, it is possible to design the sensing layer 12 (including the ssDNA of the sensor) to be porous, so that when species that do not bond to sensing layer 12 arrive to the bottom of the stack of layers of sensing device 10, they may transit sensing layer 12 toward the separation layer 14b. Separation layer 14b is placed between electret 17 and sensing layer 12, and is sized and otherwise prepared so as to collect and hold the non-bonded materials into a dump-like reservoir or reservoir layer. The collection of these materials prevents them from interfering with the measurements by affecting the dielectric constant of the material between electrodes 11, and by leaving only the bonded attached molecules to affect the capacitance of the resonator.

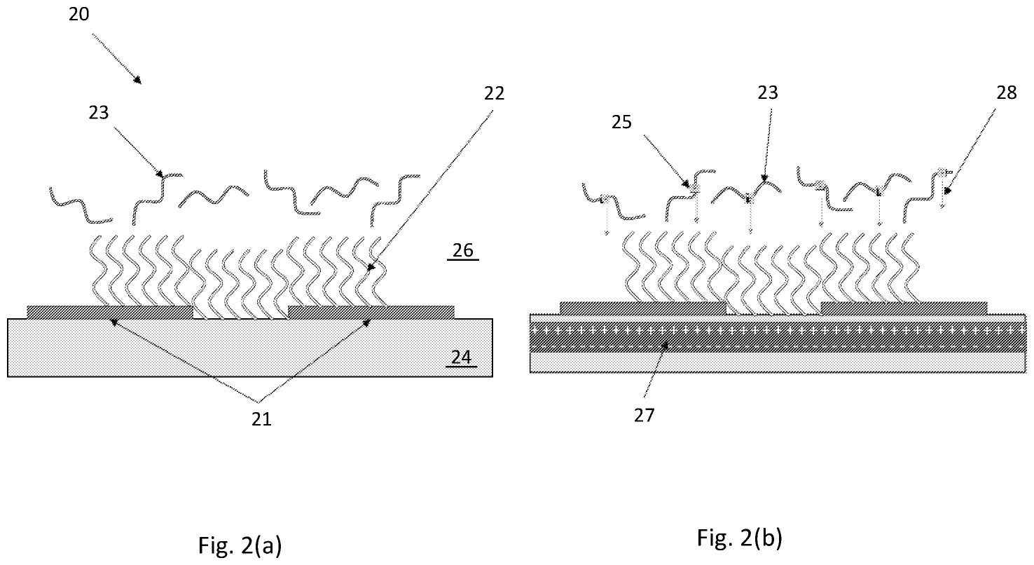

FIGS. 2A-2B illustrate a simplified version of the sensor device 10 of FIGS. 1A-1C, with particular emphasis on the action of the electret layer. As discussed above, the electret layer enables passive movement of DNA-like molecules through sensor device 10, without requiring moving parts or a power source for functioning.

FIG. 2A illustrates a sensing device 20. Sensing device 20 includes electrodes 21. The to be sensed ss-DNA material 23 is intended to be coupled to the ss-DNA material 22 which is immobilized and configured within gaps between the electrodes 21, but may also may exist at other surfaces of the electrodes or their vicinity. The sensor device components are within a medium 26, which may be, for example, a liquid, a gel, a porous material, or a liquid-impregnated paper. For simplicity of the explanation, substance 26 may be considered a liquid. In this liquid 26, many particles and/or molecules may be randomly dispersed, among them DNA-like molecules 23. Occasionally, one such molecule might arrive to the sensing zone and bond to the attached ssDNA-like molecule 22. However, this random motion generally will take a large time to occur. In turn, it will take a long time to accumulate enough ds-bonded molecules to make a discernible effect on the dielectric constant and therefore on the frequency of the sensor. Some means are required to accelerate and direct the to-be tested species 23 to enter into contact with the attached molecules 22. This is the function of electret layer 27. The principle of operation is based the fact that single-stranded DNA materials have an intrinsic negative charge spread across their extent. This is shown schematically in FIG. 2B, in which the DNA-like molecules have negatively-charged zones 25. When a positive charge is created around the sensor material, the species 23 that is to-be-tested is attracted to the positive charge and moves selectively in the preferred direction.

The term “electret” refers to a material into which a pole is introduced, such that into one of its surfaces a positive charge arises, and, if designed so, at the opposite surface a negative charge originates. In general, the electret material is prepared from a sheet of polymer by applying a high voltage to the polymer film while the film is being heated, so that the molecules shift, thus creating the charge distribution. Other materials and devices may be used to achieve the same result. For example, a single-poled material (which may be charged by a corona discharge method) with positive charges may be used to attract the negatively charged species and bring them into contact with the attached molecules in the sensing zone. There are various other ways to prepare the electret material, of various kinds which are known in the art. Some electrets may be obtained as a ready-made material and some may be prepared in situ.

A layer of electret material 27 is accordingly placed below the electrodes 21 and the gap region of the capacitor. The electret 27 is arranged parallel to the sensing zones, where the attached sensing material is expected to bond to the material to be tested. As discussed above, a separation layer may separate between the electrodes 21 and the electret 27, so long as this layer does not affect the function of the electret 27 on the region above the electrodes. This layer may be impermeable, porous, or semipermeable. The positively charged surface of the electret faces the solution 26, where the to-be-tested species 23 are spread. The to-be-tested species 23 have charged locations 25, which under the effect of the electrical field cause the molecules to move in direction 28, toward the sensing zone where the attached DNA-like molecules 22 are located. If molecules 23 and 22 match, they will bond, creating a new molecule with different dielectric constant values, thereby changing the capacitance, and signaling a positive detection.

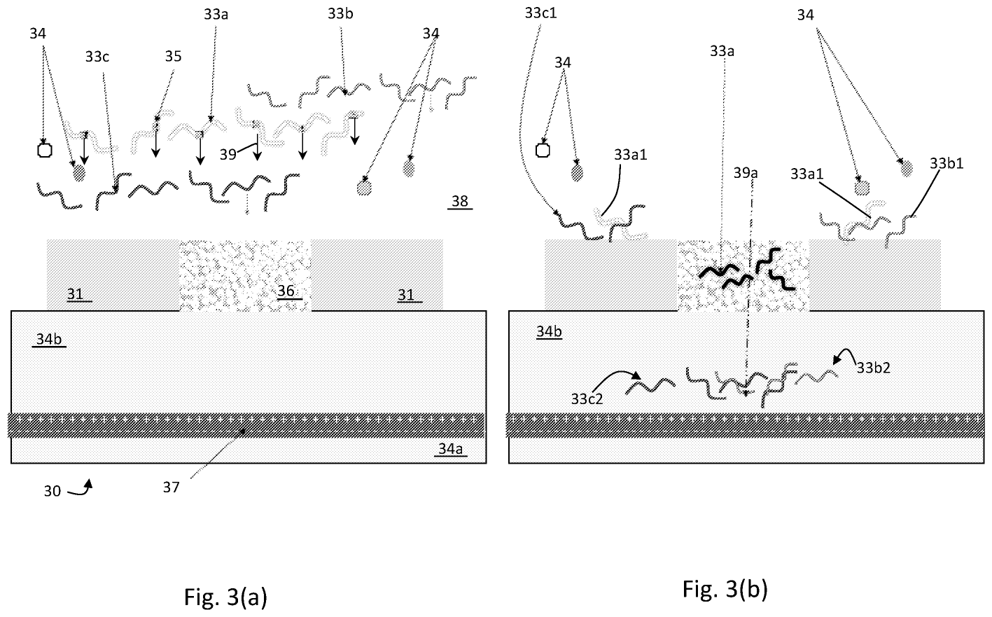

FIGS. 3A-3B illustrate an implementation of the “smart wipe” sensor device, with microbeads having single-stranded DNA attached thereto arranged between the electrodes 31. Referring to FIG. 3A, sensing device 30 includes substrate layer 34a, on top of which is electret layer 37. On top of the electret layer, a separation layer 34b is shown. The separation layer 34b is schematically shown as being relatively thick, but it can be any size. The separation layer 34b serves as a dump reservoir. On top of this layer, capacitor electrodes 31 are detailed. Between the electrodes 31, dielectric material 36 is shown composed of microbeads with ss-DNA-like fragments attached to them. One of the purposes of the use of beads is to expose additional surface area on which the ssDNA is able to attach to a surface and by this to increase the amount of material present between the capacitor plates (for example). The beads may be made of many materials. In particularly advantageous embodiments, the beads are made of conductive material, such as metal. The metal beads may be covered with a nonconductive isolation layer, in order to prevent a short-circuit between the conductive layers of different beads. The use of microbeads with DNA-like fragments attached thereto, and particularly the use of conductive microbeads in increasing the sensitivity of the capacitor electrodes, are described at length in the aforementioned priority applications, U.S. Provisional Applications No. 63/428,087and No. 63/435,258, as well as in the co-filed PCT Patent Application, Attorney Docket No. 45721/WO/22, entitled “Passive Sensor Capable of Detection of Materials Including Specific DNA or DNA-Like Strands.”

The beads are packed together forming a structure that includes voids therethrough. The structure of the voids depends on the method of placement of the microbeads and other parameters such as the density of packing. The voids might be filled with substances, such as air, gas, liquid, or gels, and preferably a substance that permits the motion of molecules therethrough. The microspheres may be packed such that molecules and other species may percolate and move from one side of the packing to another.

Above and around the layer of microspheres, chemical solutions 38 exist, for example as a liquid or gel or impregnated into a wet sheet layer, as discussed above. When the sample enters into contact with these solutions, the chain of events previously described starts to occur. As a result, the sample material is deconstituted into various species. These include DNA-like molecules, whether double-stranded, single-stranded, or single-stranded fragments, which are sketched in FIG. 3A as 33a, 33b, and 33c. These molecules have some negatively charged locations 35 on them. The solution also includes other species 34, such as cell components i.e. membrane pieces, mitochondria, etc. which are not charged or not affected by the electret. Once these components are created or released, the charged components start to move, by the effect of the electret 37, in direction 39 toward the electret 37. The electret has a positive charge toward the molecules, and therefore attracts the negatively-charged species. In addition, if there are any positively-charged species in the solution, they will move away from the positively charged electret. It is also understood by those skilled in the art, that, in an alternative embodiment not shown here, the negatively charged side of the electret may alternatively face toward the solution (opposite to what was described so far), and the species in such a case, the negative charged molecules or species will move away from the electret while the positive charged elements will be attracted and move towards the electret.

Therefore, as shown in FIG. 3B, the different species will start their motion due to the impact of the electret. Some of these molecules or charged components 33a1, 33b1, 33c1 and so forth, will move and eventually be blocked and get stuck on components of the sensors such as electrodes 31. Other charged molecules and components will go along a path 39a which traverses through the material 36, which resides between the electrodes 31. If such molecules are ssDNA-like fragments of the sample 33a, that are configured to react with the ssDNA fragments of the sensor, attached to the microbeads of material 36, then, while traversing this zone, these molecules 33a will eventually bond to the attached counterpart molecules and stop their motion and remain in the gap between the electrodes, and therefore change the value of the dielectric constant between the electrodes. This, in turn, changes the resonant frequency of a resonance circuit of which the capacitor is a part, as discussed. On the other hand, all other molecules that cannot bond to the existing material 36 will traverse the path between the microspheres and percolate all the way into spacer layer 34b, still in their motion 39a toward electret 37, making the layer where they accumulate as a dumping reservoir for the unreacted charged molecules. In addition, uncharged components 34 will not be affected by the electret and, accordingly, remain in their initial location, or drift randomly in the solution layer 38. Any positive charged species that will be repelled from the electret will remain in this layer as well. Due to this transport effect, the unwanted components will not remain in the sensing zone where dielectric material 36 is present, and only the intended molecules 33a will remain, so most of the variation in dielectric constant between the electrodes 31 is due to the effect of the intended molecules.

Those skilled in the art may understand additional variations are possible, such as using more than one solution layer 38, or using one or more spacing layers 34b, to collect or treat the dumped molecules. Also, the packed material 36 can be microbeads, but it can be of any other structure or material that allows the required species to stop moving in the area of interest and all the other species to pass through towards the dumping reservoir zone.

Referring now to FIGS. 4A-B, systems and methods for manufacture of a smart wipe sensing device are disclosed. As discussed, in the smart wipe embodiment, the sensing device is constructed of several layers of materials, part of them patterned, part of them conductive, and part of them capable of holding liquids. As mentioned, this device may be fabricated of rigid material layers or of flexible materials. One fabrication method suitable for implementing such a material, at low-cost, is the “Roll to Roll” fabrication technology. This is a method known to those skilled in the art, that resembles printing of newspapers or the methods to fabricate wet wipes and many other consumer articles made of sheets of papers, sheets of clothing or woven or non-woven fabric materials. The method, as its name suggests, takes a roll of material, such as a paper or a fabric, and processes it through various stages and spins it into a second roll, where it is remains either completed as a product or is ready to be cut into small pieces for selling.

In FIG. 4A, it is possible to see the entire conceptual “Roll to Roll” fabrication complex. Starting from the left, a roll of substrate material 40 is unwound and starts its journey running to the right (as shown by the arrow). At a different place, a roll of an electret material 41 is unwounded and passed through a mold 41a, which cuts the electret sheet in the form and in the relative location where the electret layer should be placed. At this point, the electret material is not yet poled. The rest of the sheet of electret material is wound again on roll 41c. The electret piece is temporarily attached to roll 42, which transfers the electret piece 41b towards the substrate sheet running forward. When the electret is between roll 42, sheet 40a and second roll 42a, the electret piece 41c is laminated onto the substrate layer 40a. Subsequently, the electret enters a zone 41d, where it is heated and an electric field is applied, thereby poling the electret, with one side having a negative charge and the other side a positive charge. As mentioned, this is one method for poling or charging the electret or charged layer, but other methods also might be used, such as corona discharge. The poled electret 41e proceeds to the next fabrication station. At this step, a spacer material 43 is laminated onto the electret layer, as shown in 43a. Meanwhile, roller 44 is used at the conductor fabrication station 44a. At this station, using various possible methods known in the art, the sensing layer is fabricated. The conductors of the sensing layer may be fabricated either by evaporation of a metal layer through a patterned mask or etching of a metal-covered or deposited sheet through a defined mask or applying a conductive paint or paste, or any other suitable method for generating conducting patterns onto the roll. The metal circuit 44b is transferred as layer 44c by means of roller 44b, which ends up laminating the pattern 44e and creating the layered sensing device 44f, with the conducting structures on it.

It should be understood that the use of the rollers mentioned is one possible option of many possible configurations of the rollers and the sheets moving. Alternative processes include patterning the conducting layer directly on the moving stacking sheet, or the use of more or fewer rollers to transfer the pattern, as well as rollers or other components that may be useful to assist or enhance the fabrication of the conduction layer or layers.

Subsequently, a sealing layer from a roller 45 seals the conducting layers as shown at 45a. In order to expose the active zone of the sensor around the capacitor electrodes zone, an opening 45c is cut. Opening 45c can be made by various methods, either by pre-patterning the sheet 45, or removing the material at the required location in a method such a laser beam 45b, that ablates the sheet material and makes the opening. At this step, most of the device layers are completed, as illustrated at 45d, whereby the substrate layer, the spacer layer, the electret and conducting structures, and the active region opening are done.

At this stage, the biological material, such as the ssDNA or the cDNA, is attached to the active zone in the opening that was prepared for this purpose. To perform this task, several conceptual possibilities are presented. According to FIG. 4a, the biological material attachment is outsourced. The fabricated device at this staged is rolled in roll 45e, and sent to an appropriate fabrication facility to prepare the biological material in the patterned openings. In return from the biological plant, the roll 46 is unwound in to the production tool. This time, the devices 46a already contain the ssDNA on the correct location in the openings to the active layer. The device advances to station 46c, where if required, a liquid or other substance 46b is poured into the sheet openings and restricted by a doctor blade 46d. The thus fabricated device 46e is almost ready and needs to be sealed. This occurs at the next station, where a sheet of material 47 which contains all the chemicals and solutions to release, cut and separate the DNA-like fragments are soaked in it. The sheet layer is attached to the other layers, thus sealing and completing the device 47a. The next step that might be done at this or a later time is to cut the large roll of the thus fabricated wipe into smaller pieces 48, as might be preferable for packaging, transportation, storage and selling.

It should be emphasized that this is a conceptual explanation of the fabrication sequence and those skilled in the art may understand how to implement this for different practical cases. In particular, rollers shown in the description might not be required for some implementations, while other elements and rollers might be added as required.

FIG. 4B illustrates another option for application of the biological material. Compared to the process illustrated in FIG. 4A, most of the steps remain the same up to where component 45d is ready, meaning that all the layers including the electret material, the conductors, the separators, and the sealing layers, have been prepared and the active area is cut open. However, instead of rolling the sheet to be outsourced for application of biological material, the biological material, including beads 49 onto which they are attached, are brought to the fabrication line. The beads 49 are poured into the respective opening and set in the opening, optionally using a doctor blade 46d. At the next station a sheet of solution layer material 47, which had been soaked in and contains all the chemicals and solutions to release, cut and separate the DNA-like fragments, is attached to the other layers, thus sealing and completing the device 47a. The next step that might be done at this or a later time, is to cut the large roll of the thus fabricated wipe, into smaller pieces 48, as might be preferable for packaging, transportation, storage and selling.

What is claimed is:

1. A multilayered sensor device for detecting DNA or DNA-like strands within a biological sample, comprising:
a substantially planar electret layer, the electret layer having a positive pole that is configured to attract negatively charged particles;
a sensing layer configured above the electret layer, the sensing layer including a plurality of sensors comprising pairs of conductive elements with gaps therebetween, wherein strands of single-stranded DNA of the sensor are configured within the gaps, wherein the strands of single-stranded DNA of the sensor within the gaps are configured to couple with strands of single-stranded DNA from the biological sample; and
a solution layer configured above the sensing layer, said solution layer comprising chemical solutions for breaking down a biological sample into DNA strands;
wherein the positive pole is arranged relative to the sensing layer and solution layer such that, following breakdown of the biological sample into DNA strands in the solution layer, the positive pole of the electret layer draws strands of single-stranded DNA of the sample through the solution layer and to the sensing layer.

2. The multilayered sensor device of claim 1, further comprising a separation layer between the sensing layer and the electret layer.

3. The multilayered sensor device of claim 2, wherein the separation layer is permeable to liquids and comprises a dump reservoir for liquids that are attracted to the electret layer but are not coupled to the single-stranded DNA of the sensor.

4. The multilayered sensor device of claim 1, wherein each pair of conductive elements comprises plates of a capacitor, with the single-stranded DNA of the sensor configured between the plates of the capacitor.

5. The multilayered sensor device of claim 4, wherein the single-stranded DNA of the sensor is arranged on conductive beads configured between the plates of the capacitor.

6. The multilayered sensor device of claim 5, wherein the conductive beads are arranged with voids therebetween, to permit passage therethrough of biological material from the sample that does not couple to the single-stranded DNA.

7. The multilayered sensor device of claim 6, wherein the voids are filled with one or more of air, gases, liquid, or gels.

8. The multilayered sensor device of claim 1, wherein each pair of conductive elements comprises optical waveguides, wherein the single-stranded DNA of the sensor is arranged in gaps between the waveguides.

9. The multilayered sensor device of claim 1, further comprising a substrate layer arranged below the electret layer, wherein the substrate layer is non-conductive and liquid-resistant.

10. The multilayered sensor device of claim 9, further comprising a sensor sealing layer configured between the sensing layer and the solution layer, the sensor sealing layer comprised of material that is impervious to liquids and including a central opening therein opposite the single-stranded DNA of the sensing layer.

11. The multilayered sensor device of claim 10, wherein the opening is filled with one or more of a non-conducting liquid, a gel, or a compressed powder.

12. The multilayered sensor device of claim 1, wherein the solution layer comprises a plurality of sheets stacked one over the other, with each sheet containing a plurality of solutions configured to perform specific chemical functions on the biological material.

13. The multilayered sensor device of claim 12, wherein the plurality of sheets includes an uppermost layer containing a protein kinase solution; a second layer below the uppermost layer containing a restriction enzyme solution, and a third layer below the second layer containing a dissociating solution.

14. A method of drawing single-stranded DNA strands to a sensor for sensing presence of specific single-stranded DNA strands, wherein the sensor comprises a substantially planar electret layer, the electret layer having a positive pole that is configured to attract negatively charged particles; a sensing layer configured above the electret layer, the sensing layer including a plurality of sensors comprising pairs of conductive elements with gaps therebetween, wherein strands of single-stranded DNA of the sensor are configured within the gaps, wherein the strands of single-stranded DNA of the sensor within the gaps are configured to couple with strands of single-stranded DNA from the biological sample; and a solution layer configured above the sensing layer, said solution layer comprising chemical solutions for breaking down a biological sample into DNA strands; and the method comprises:
breaking down the biological sample into single-stranded DNA of the sample in the solution layer; and
drawing the single-stranded DNA of the sample from the solution layer to the sensing layer with the positive pole of the electret layer.

15. The method of claim 14, further comprising coupling strands of single-stranded DNA from the sample with strands of single-stranded DNA of at least one sensor, and measuring resulting changes of electrical properties of the at least one sensor.

16. A method of manufacturing a multilayered sensor device, comprising:
providing a substrate layer;
cutting a plurality of sheets of electret material, and laminating the plurality of sheets of electret material onto the substrate layer to thereby form a plurality of electret layers;
heating and applying an electric field to each electret layer to thereby pole the electret material;
laminating a sensing layer onto each separation layer, the sensing layer including a plurality of pairs of conductive elements with gaps therebetween;
configuring strands of single-stranded DNA of the sensing layer within the gaps; and
laminating a solution layer above the sensing layer, the solution layer comprising chemical solutions for breaking down a biological sample into DNA strands of the sample.

17. The method of claim 16, wherein the step of providing a substrate layer comprises providing the substrate layer in the form of a roll and unrolling the roll, and wherein the laminating steps are performed on the unrolled roll.

18. The method of claim 16, further comprising performing each of the laminating steps using a roll-to-roll process.

19. The method of claim 16, wherein each laminating step comprises laminating respective layers of multiple multilayered sensor devices simultaneously, and further comprising, following completion of the laminating steps, separating the multilayered sensor devices.
